(19) United States
(12) Patent Application Publication
(10) Pub. No.: US 2020/0041513 A1
(43) Pub. Date: Feb. 6, 2020

(54) RESIDUAL DISEASE DETECTION IN MULTIPLE MYELOMA

(71) Applicants: HORIBA ABX SAS, Montpellier (FR); CENTRE HOSPITALIER UNIVERSITAIRE DE MONTPELLIER, Montpellier (FR); INSTITUT NATIONAL DE LA SANTE ET DE LA RECHERCHE MEDICALE (INSERM), Paris (FR); UNIVERSITE DE MONTPELLIER, Montpellier (FR)

(72) Inventors: Guilhem REQUIRAND, Carnon (FR); Bernard KLEIN, Saint Clément de Rivière (FR); Thierry REME, Sainte Croix de Quintillargues (FR); Jérôme MOREAUX, Montpellier (FR); Sébastien RAIMBAULT, Argelliers (FR); Philippe NERIN, Assas (FR); Rosalie PLANTEFEVE, Montreal (CA); Elina ALATERRE, Montarnaud (FR); Jean-Michel GARCIA, Grabels (FR)

(21) Appl. No.: 16/303,598

(22) PCT Filed: May 22, 2017
(86) PCT No.: PCT/EP2017/062307

§ 371 Date: Nov. 20, 2018

(30) Foreign Application Priority Data
May 20, 2016 (FR) 1654514

(51) Int. Cl. G01N 33/574; G01N 15/14; G01N 33/50

(52) U.S. Cl. CPC G01N 33/574; G01N 15/14; G01N 2333/70596; G01N 2333/70578; G01N 33/5094

(57) ABSTRACT

The invention relates to a method for detecting, by means of flow cytometry, the presence of normal plasma cells and tumoral plasma cells in a sample of cells from a patient.

The present invention lies in the field of diagnosing or assisting diagnosing and monitoring minimal residual disease in malignant hemopathies and the subject thereof is a method for detecting tumoral plasma cells and a method for diagnosing or assisting diagnosing and monitoring monoclonal gammopathies.

Multiple myeloma (MM) or Kahler's disease is a malignant hemopathy characterized by the accumulation of tumoral plasma cells (B lymphocytes) in the bone marrow, and the accumulation of a whole monoclonal immunoglobulin or of a monoclonal light chain. This pathological condition affects 2000 new patients per year in France, 19 000 in Europe and 19 000 in the United States. The median survival expectancy of patients 65 years old or younger is 6-7 years. The diagnosis of MM includes bone signs, an increased sedimentation rate, the presence of monoclonal immunoglobulin and the use of a myelogram showing:
the presence of dystrophy plasma cells (normal cytoplasm but nuclear cytological abnormalities (multinuclear cells, multiple or atypical mitosis)); and
plasmacytosis, namely either the existence of a plasmacytosis greater than or equal to 10% (minor criterion), or, in certain cases, a plasmacytosis greater than 30% (major criterion, severe pathological condition).

MM is in all cases preceded by a benign monoclonal gammopathy (MGUS from Monoclonal Gammopathy of Undeterminated Significance). MGUSs are detected in 2 to 4% of subjects 60 years old, 10% of subjects 70 years old, 20% of subjects 80 years old. MGUSs progress to an MM with a degree of transformation of 1% per year, independent of the date of diagnosis of the MGUS.

MM represents 1% of new cases of cancer per year, 10 to 15% of malignant hemopathies and is the second most common malignant hemopathy after non-Hodgkin's lymphoma. Furthermore, the costs associated with its treatment are among the highest.

Recent data from the literature show that the identification and characterization of tumoral and normal plasma cells in patients suffering from MM provide a reliable marker for the response to treatment. The group of Prof. San Miguel (Paiva et al., 2014) has shown that, when there exists fewer than 1 tumoral plasma cell per 10 000 total leukocyte cells (with a sensitivity of 10⁻⁴) three months after a high-dose chemotherapy followed by a hematopoietic stem cell autograft, the patients have a significantly increased survival. The tumoral plasma cell/normal plasma cell ratio is a prognostic factor independent of the usual prognostic markers (Mateo et al., 2008). The proliferation index of tumoral plasma cells, calculated by flow cytometry, is also a powerful prognostic factor. It can be decisive for evaluating whether or not a patient is in active relapse. The median level of S-phase cells is 1% in an individual who is ill (Paiva et al., 2012) and is increased in the patients who are in relapse.

Moreover, minimal residual disease (MRD) in multiple myeloma denotes the persistence of residual tumor cells after treatment. Plasma cells represent up to 5% of the cells in the bone marrow, and the presence of MRD is defined by the presence of at least 50 tumoral plasma cells per 500 000 total leukocyte cells, i.e. 0.01% of the cells (Rawstron et al., 2013).

The median survival time of a patient is 7 months without treatment, from 3 to 4 years with conventional chemotherapy treatment and from 6 to 7 years with a treatment with high-dose chemotherapy and autograft. Thus, the monitoring of MRD after treatment is also very important in order to detect relapses as early as possible and to improve the therapeutic treatment.

At the current time, there are various methods for detecting MRD, for example “Deep Sequencing”, quantitative PCR (Polymerase Chain Reaction) with specific primers and multiparametric flow cytometry (MFC) Martinez Lopez et al., 2014; Paiva et al., 2008). The first two methods have a high sensitivity; however, they remain very expensive, and lengthy, and are difficult to automate. Thus, multiple color flow cytometry is at the current time the most suitable method for evaluating patient response, for detecting relapse early on and for improving the therapeutic treatment in order to maximize the treatment benefit/cost ratio.

Given the very small amount of normal plasma cells present in the bone marrow, i.e. a median value of 0.44% mononuclear cells in a healthy donor, this means that a large number of cell events must be acquired in order to minimize statistical errors.

Currently, a method set up by the STI laboratory (Caraux et al., 2010, 2012) takes 2 days, i.e. 16 working hours, in order to provide clinicians with a result. Furthermore, such a method uses at least seven colors, seven detectors, two lasers and five tubes, and therefore a large and expensive number of devices and reagents. In addition, the use of such a method requires the knowhow of an expert in cytometry for the preparation, acquisition, compensation and analysis of the data.

There is therefore a real need to develop new methods and compositions for the diagnosis and monitoring of the progression of this disease, and in particular to improve the sensitivity and the reliability of the tests for detecting tumoral plasma cells, while at the same time reducing the costs and the complexity of the various experimental steps.

The inventors have developed a method for detecting tumoral plasma cells which has very high sensitivity, reproducibility and reliability.

The method according to the invention makes it possible, through the use of a particular combination of tumoral and normal plasma cell markers, to obtain results from detection of normal and tumoral plasma cells that are at least equivalent to those obtained using the detection methods already known in the prior art.

Compared with the prior art methods, the method according to the invention has the advantage of being able to be carried out in a single container, such as a tube or a well of a plate with wells, comprising the sample to be analyzed and the various markers. This has in particular the advantage of being less expensive, of reducing the time and the complexity of the various experimental steps, and of reducing the delay in obtaining the results. Thus, the method according to the invention makes it possible to decrease the requirements for qualified personnel and the associated risks of errors, thus at the same time increasing the reliability of the method. Furthermore, the decrease in complexity for various experimental steps of the method according to the invention makes it possible to improve the reproducibility and to increase the productivity.

The subject of the invention is a method for detecting, by flow cytometry, the presence of normal plasma cells and of tumoral plasma cells in a sample of cells from a patient, said method comprising the following steps:

a) bringing said sample of cells into contact with:
a κ chain marker, a λ chain marker, and a marker chosen from: a CD38 marker and a CD138 marker, each of the three markers emitting a different signal (hereinafter the “first three signals”), and
one or more tumoral plasma cell marker(s), each of these one or more marker(s) emitting a signal, in particular one and the same signal, otherwise known as “single detectable signal” (hereinafter the “fourth” signal), different than the first three signals, and
one or more normal plasma cell marker(s), each of these one or more marker(s) emitting a signal, in particular one and the same signal otherwise known as “single detectable signal” (hereinafter the “fifth signal”), different than the first four signals,
   b) detecting the plasma cells characterized by:
a positive signal, in particular a high positive signal, with a CD38 marker or a CD138 marker, and
a positive signal, in particular a high positive signal, with a κ chain marker or a λ chain marker,
   c) detecting the tumoral plasma cells among the plasma cells detected in step b), having at least one of the following criteria (the other plasma cells being considered to be normal):
a positive signal with at least one marker specific for tumoral plasma cells, and/or
a negative or significantly reduced signal with at least one marker for normal plasma cells,
   d) analyzing the results obtained during steps b) and c) and quantifying the normal plasma cells and the tumoral plasma cells.

According to other embodiments, use may be made, as an alternative to the respective CD38 and CD138 markers, of one or more markers chosen from the group consisting of: a CD229 marker, a CD54 marker, a CD319 marker (Pojero et al., Utility of CD54, CD229, and CD319 for the identification of Plasma Cells in Patients with Clonal Plasma Cell Diseases), a CD319 marker, or a CD269 marker (Frigyesi et al., Blood. 2014 Feb. 27; 123(9):1336-40. doi: 10.1182/blood-2013-09-529800). Mention may also be made of a MUM1/IRF4 marker which is an intracytoplasmid marker.

The abovementioned markers can be used individually, as a replacement for the respective CD38 and CD138 markers, or in combination with them and/or in particular with CD38.

A κ chain marker, a λ chain marker and a CD229 marker may thus for example be used.

It is understood from the remainder of the description and the examples illustrating the invention which are described hereinafter that the CD marker(s) according to the invention, for one and the same group of markers having a common expression differential (decrease or loss versus increase or gain), are coupled or conjugated to one and the same fluorochrome, or chromophore.

According to the present invention, firstly the tumoral plasma cell marker(s) in step a) emit a (fourth) signal different than the first three signals, in the sense that it is one and the same signal different than the first three signals, in other words a common signal or a “single detectable signal” different than the first three signals.

According to the invention, the term “one and the same signal” or “single detectable signal” is intended to mean that the tumoral plasma cell marker(s) are characterized in that they emit a common signal recognized by the detector.

According to one preferred embodiment, said tumoral plasma cell marker(s) are coupled or conjugated to the same fluorochrome so as to emit one and the same signal otherwise known as “single detectable signal”, different than the first three signals.

According to one alternative, said tumoral plasma cell marker(s) are coupled conjugated to fluorochromes which emit signals that can be detected, in frequency bands close to, equivalent to or that can be likened to one and the same signal otherwise known as “single detectable signal”, by a detector of suitable frequency, said single detectable signal being different than the first three signals.

According to the present invention, secondly, the normal plasma cell marker(s) in step a) emit a (fifth) signal different than the first four signals, in the sense that it is one and the same signal different from the first four signals, in other words a common signal or a “single detectable signal” different that the first four signals.

According to the invention, the term “one and the same signal” or “single detectable signal” is intended to mean that the normal plasma cell marker(s) are characterized in that they emit a common signal recognized by the detector.

According to one preferred embodiment, the normal plasma cell marker(s) are coupled or conjugated to the same fluorochrome so as to emit one and the same signal otherwise known as “single detectable signal”, different than the first four signals.

According to one alternative, said normal plasma cell marker(s) are coupled or conjugated to fluorochromes which emit signals that are detectable, in frequency bands close to, equivalent to or that can be likened to one and the same signal otherwise known as “single detectable signal”, by a detector of suitable frequency, said single detectable signal being different than the first four signals.

The detection of one and the same signal for one and the same group of markers having a common expression differential (decrease or loss versus increase or gain), advantageously makes it possible to reduce the number of fluorochromes and/or of detectable signals, and therefore the number of detectors and of sources in the detection method according to the invention, for a rapid (smaller data files) and simplified analysis.

Preferentially, the plasma cell marker according to the invention is a plasma cell antigen marker, preferentially a plasma cell cluster of differentiation (CD) marker.

The term “marker” is intended to mean any molecule(s) enabling the recognition of the target and the detection thereof by the emission of a detectable signal. Advantageously, for a peptide sequence labeling, the markers according to the invention are aptamers or antibodies conjugated to fluorochromes, preferentially the fluorochromes are selected from PE-Cy5.5, PE-CF594, PE-Cy7, PE, APC and FITC. Alternatively, for a DNA labeling, the markers according to the invention can also be base analogs, autofluorescent molecules when they bind to or intercalate into DNA (for example DAPI; blue fluorescence at approximately 456 nm) or molecules conjugated to fluorochromes (for example antibodies) which can bind to or intercalate into DNA. Advantageously, according to the invention, the tumoral plasma cell marker(s) are conjugated to one and the same fluorochrome (chromophore) for the emission of one and the same signal, distinct from the other signals. In addition, the normal plasma cell marker(s) are conjugated to another fluorochrome (chromophore) for the emission of one and the same signal, distinct from the other signals.

This makes it possible to obtain one and the same signal per group of markers having a common expression differential (decrease or loss versus increase or gain), simplifying the method and advantageously allowing implementation in a single container.

Thus, the amount of signal emitted by a marker according invention is linked to the amount of marker/target complexes, and therefore to the level of expression of peptide sequences and/or to the amount of target DNA.

The term “signal” is intended to mean any signal emitted with properties which make it possible to recover this signal after transmission. These properties can for example be frequencies, modulations, encodings. Preferentially, the detectable signals are emitted in frequency bands which they are assigned, more preferentially colors of the visible spectrum in an optical transmission. Thus, various markers which emit different detectable signals in close frequency bands can be detected by one and the same detectors, or different markers which emit one and the same single detectable signal can be detected by one and the same detector.

According to one particular preferred embodiment, use will be made, according to the invention, of one or more markers which emit one and same signal otherwise known as “single detectable signal” or one and the same group of markers having a common expression differential (decrease or loss versus increase or gain), distinct from the other signals.
One or the same fluorochrome (chromophore) thus corresponds to each group of markers having a common expression differential (decrease or loss versus increase or gain), so as to give the emission of a single signal.

The term “detector” is intended to mean any system/device which makes it possible to recover one of the detectable signals among the others, the detection thereof and/or the quantification thereof. Preferentially, the detector uses a filter to isolate the signal that it detects. More preferentially, this filter is of band-pass type. Preferentially, the detection and/or quantification is carried out by a photonic/electronic converter, for example photodiodes, avalanche photodiodes, photomultipliers (PMTs).

The term “normal plasma cell” is intended to mean plasma cells, plasmatic cells or plasmatocytes having the (cluster of differentiation; CD) antigens chosen from CD19, CD27, CD38, CD45, CD81, CD5L, CD11a, CD16b, CD24, CD36, CD52, CD68, CD79a, CD80, CD82, CD84, CD99, CD138, CD163, and not having the CD20, CD28, CD52, CD56, CD117 and CD200 antigens.

Normal plasma cells exhibit the kappa or lambda intra-cytoplasmic chains with respective proportions of ⅔ and ⅓.

The term “tumoral plasma cell” is intended to mean plasma cells, plasmatic cells or plasmatocytes having at least one modification relative to the normal profile:
loss of at least one CD among CD19, CD27, CD45, CD52 and CD81,
gain of at least one CD among CD20, CD28, CD56, CD117, CD200 and CD229,
change in expression level of at least one CD among CD5L, CD11a, CD16b, CD24, CD36, CD52, CD68, CD79a, CD80, CD82, CD84, CD99, CD138 and CD163, underexpressed in tumoral plasma cells, CD1D, CD2BP2, CD32c, CD47, CD59, CD109, CD138, CD300a and CD320, overexpressed in tumoral plasma cells and/or
modification of the kappa/lambda ratio (monoclonality).

The expression “positive signal” signifies according to the invention that the signal is said to be positive when the plasma-cell-specific markers used bind to their targets and emit a significantly detectable signal. The “positive signal” thus shows that the plasma cell(s) detected in step b) of the method according to the invention has(have) a significantly increased level of expression of the molecule, recognized by the marker(s) used, by the plasma cell(s) detected. A positive signal for a “CD” marker is commonly expressed by “CD⁻” or by the terminologies “CD^{low or dim}”, “CD^{mid or intermediate}” or “CD^{high or bright}” depending on the level of expression of said marker detected. Reference will also be made to a low or dim, mid or intermediate, or high or bright positive signal.

The fluorescence intensity observed on matrices such as those illustrated in the examples hereinafter, with, on the x-axis, the fluorescence intensity measured, is usually classified according to the following orders of magnitude (decades), by way of illustration:
less than 10²: negative signal, or “−”;
from 10² to 10³: low or dim positive signal, or “+/−”;
from 10³ to 10⁴: mid or intermediate positive signal, or “+”;
greater than 10⁴: high or bright positive signal, or “++”.
   According to the invention, and as illustrated in the examples and figures hereinafter, focus is in particular on the detection on a high positive signal for the detection of the tumoral plasma cells in step b) of the detection method according to the invention. This step b) of detecting the (total) plasma cells is characterized by: a high positive signal with a CD38 marker or a CD138 marker, a high positive signal with a κ chain marker or a λ chain marker. This may also be expressed by the terminologies CD38⁺⁺/κ⁺⁺ or CD138⁺⁺/λ⁺⁺ or for example CD38^{high}/κ^{high} or CD138^{high}/λ^{high}. This step b) of detecting a high positive signal of the (total) plasma cells overexpressing a CD38 or CD138 marker and a κ chain marker or a λ chain marker is advantageous in that it makes it possible to dispense with additional steps aimed at eliminating other cell types, such as B lymphocytes. The detection method according to the invention is thus simplified compared with the current detection methods.

The term “negative signal” is intended to mean that the signal is said to be negative when the plasma-cell-specific markers used do not bind, or bind weakly, to their targets and therefore do not emit a significantly detectable signal. The “negative signal” of these specific markers thus shows that the plasma cell(s) detected in step b) of the method according to the invention have a negative or significantly reduced level of expression of the molecule recognized by the marker(s) used. A negative signal for a “CD” marker is commonly expressed “CD”.

Preferentially, the cell sample used for the method according to the invention is chosen from, or obtained from, bone marrow, blood, serum, a blood extract, PBMCs (Peripheral Blood Mononuclear Cells), cerebrospinal fluid, pleural fluid and lymph nodes, preferentially the bone marrow of a patient.

According to one particular embodiment, the cell sample of the method according to the invention comes from a patient suffering from monoclonal gammopathy, or from an individual who may be suffering from a monoclonal gammopathy.

According to one preferred embodiment, the CDs specific for normal plasma cells of the method according to the invention are chosen from: CD19, CD27, CD45, CD81, CD5L, CD11a, CD16b, CD24, CD36, CD52, CD68, CD79a, CD80, CD82, CD84, CD99 and CD163, preferentially CD19, CD27, CD45 and CD81, more preferentially CD19, CD27.

According to one preferred embodiment, the CDs specific for tumurol plasma cells of the method according to the invention are chosen from: CD56, CD117, CD200, CD20, CD28, CD1D, CD2BP2, CD32c, CD47, CD59, CD109, CD229, CD300a and CD320, preferentially CD56, CD117, CD200, CD20, and CD28, more preferentially CD56, CD117, CD200.

Preferentially, the method according to the invention also comprises analyzing the FSC (Forward Scatter) and SSC (Side Scatter) signals. FSC and SSC correspond to simultaneous measurements carried out by flow cytometry making it possible to determine the physical and biological characteristics of isolated cells. By definition, the FSC corresponds to the intensity of the light, from the excitation source scattered in the axis (angle <12°), which is proportional to the size of the cell, while the SSC corresponds to the intensity of the light that is orthogonally scattered, termed “wide angle” (close to 90°), relative to the incident light source, which is representative of the intracellular complexity.

According to one preferred embodiment, the sample of the method according to the invention is brought into contact with:
a κ chain marker, a λ chain marker and a CD38 or CD138, preferentially CD38, marker, each of the three markers emitting a different signal, and
one or more marker(s), preferentially at least two CD markers of normal plasma cells, selected from the CD19, CD27, CD45, CD81, CD5L, CD11a, CD16b, CD24, CD36, CD52, CD68, CD79a, CD80, CD82, CD84, CD99 and CD163 markers, preferentially the CD19, CD27, CD45 and CD81 markers, more preferentially a CD19 marker and a CD27 marker, each of these one or more marker(s) emitting a signal, in particular one and the same signal otherwise known as “single detectable signal”, different than the first three signals, and
one or more CD marker(s), preferentially at least two CD markers, more preferentially at least three CD markers for tumoral plasma cells, selected from the CD56, CD117, CD200, CD20, CD28, CD1D, CD2BP2, CD32c, CD47, CD59, CD109, CD229, CD300a and CD320 markers, preferentially the CD56, CD117, CD200, CD20 and CD28 markers, more preferentially a CD56 marker, a CD117 marker and a CD200 marker, each of these one or more marker(s) emitting a signal, in particular one and the same signal otherwise known as “single detectable signal”, different than the first four signals.
   According to one particular embodiment of the invention, the sample of the method according to the invention is brought into contact with:
   (i) a number of markers less than ten markers, in particular less than or equal to nine markers, or even less than or equal to eight markers, or even less than or equal to seven markers, or even less than or equal to six markers with:
at least three markers for total plasma cells with a κ chain marker, a λ chain marker and a CD38 or CD138 marker, and
at least one marker for normal plasma cells, and
at least one marker for tumoral plasma cells, and
optionally at least one cell proliferation marker,
   and
   (ii) a number less than eight fluorochromes, in particular less than or equal to seven fluorochromes, or even less than or equal to six fluorochromes, or even equal to five fluorochromes, with:
three fluorochromes for the markers for total plasma cells, and
a fluorochrome distinct from the preceding fluorochromes, for the marker(s) for normal plasma cells, and
a fluorochrome for the marker(s) for tumoral plasma cells, and
optionally a fluorochrome for the cell proliferation marker(s).
According to one particular and preferred embodiment of the invention, the method according to the invention is carried out in a single container, such as a tube or a well of a plate with wells, comprising the sample to be analyzed and the various markers. Preferably, the method according to the invention is carried out in a tube.
   Preferentially, the sample of the method according to the invention is brought into contact with:
   a κ chain marker, a λ chain marker and a CD38 marker, each of the three markers emitting a different signal, and
   a CD19 marker and a CD27 marker, emitting a signal, in particular one and the same signal otherwise known as “single detectable signal”, different than said first three signals, and
   a CD56 marker, a CD117 marker and a CD200 marker, emitting a signal, in particular one and the same signal otherwise known as “single detectable signal”, different than the first four signals.

Preferentially, the CD38 or CD138 marker is respectively an anti-CD38 or an anti-CD138 antibody conjugated to the PE-Cy5.5 fluorochrome, the κ chain marker is an anti-κ antibody conjugated to the PE-CF594 fluorochrome and the λ chain marker is an anti-λ chain antibody conjugated to the FITC fluorochrome; the detection being carried out on the appropriate flow cytometry detectors according to the prior art.

Advantageously, the CD19 and CD27 markers are anti-CD19 and anti-CD27 antibodies conjugated to the PE-Cy7 fluorochrome, and the CD56, CD117 and CD200 markers are anti-CD56, anti-CD117 and anti-CD200 antibodies conjugated to the PE fluorochrome; the detection being carried out on the appropriate flow cytometry detectors according to the prior art.

Furthermore, as previously indicated, the proliferation index of the tumoral plasma cells corresponds to the percentage of tumor cells in the S phase, calculated by flow cytometry, and is a powerful prognostic factor, making it possible to evaluate the speed of development of the tumor mass and to determine whether or not a patient is in active relapse.

Thus, according to one preferred embodiment, the method according to the invention also comprises determining the proliferation index of the tumoral plasma cells, comprising the following steps:
A. bringing said cell sample into contact with one or more cell proliferation marker(s), preferentially two cell proliferation markers, emitting a signal different than the other markers used;
B. detecting the proliferating tumor plasma cells by analysis of the signal emitted by said marker(s);
C. determining the percentage of tumor cells and of normal plasma cells which are in the S phase;
D. optionally determining the diploidy (G0/G1 or G2/M) when at least one of said cell proliferation marker(s) is a DNA marker.

The term “proliferation markers” is intended to mean any markers which make it possible to determine whether a cell is in the G0/G1, G2/M or S cell cycle phase. The proliferation markers are for example chosen from:
proliferation markers which make it possible to identify the S-phase cells, such as base analogs of BrdU or EdU type,
proliferation markers which make it possible to identify the G0-phase cells (quiescent cells) or G1/S/G2/M-phase cells (non-quiescent cells), for example markers for proteins such as Ki67 (Dako),
proliferation markers which make it possible to identify the G0/G1-phase or G2/M-phase cells, for example deoxyribonucleic acid (DNA) markers, such as acridine orange, DAPI, propidium iodide (PI), or Hoechst dye (33342 or 33258).

According to one particular and preferred embodiment, use will be made of at least one proliferation marker which makes it possible to identify the S-phase cells, such as base analogs of BrdU or EdU type, and preferably of BrdU type.

According to one particular embodiment, the markers for tumoral plasma cell proliferation of the method according to the invention are selected from:
base analogs of BrdU type recognized by an anti-BrdU antibody conjugated to allophycocyanin (APC) or ethynyldeoxyuridine (EdU) recognized by an azide coupled to a fluorochrome, preferentially Alexa Fluor® 647; and/or
proteins which are present in the active cell cycle, for example Ki67 recognized by an anti-Ki67 antibody conjugated to a fluorochrome, preferentially Alexa Fluor 647; and/or
deoxyribonucleic acid (DNA) markers, such as DAPI, PI, Hoechst dye (33342 or 333258), etc.

The term “BrdU” (BrdU FlowKit; BD Pharmingen) is defined as a synthetic nucleoside, which is a structural analog of thymidine, which can be incorporated into DNA and which can be detected by a specific antibody for determining the phase of the cell cycle for a cell and in particular whether said cell is in the DNA replication phase (S phase). The term “EdU” (Click-iT® EdU; Thermofisher) is defined as a synthetic nucleoside (structural analog of thymidine) which is incorporated into DNA during its replication. The bonding between an alkyne of the ethynyl fragment of EdU and an azide coupled to a fluorochrome is carried out by the cycloaddition reaction, one of the most popular ligation reactions in click-chemistry. The EdU can then be detected when it is incorporated into the DNA in order to determine whether the cells of interest are in the DNA replication phase (S phase).

Advantageously, the method for determining the proliferation index of the tumoral plasma cells according to the invention comprises the following steps:
A1. bringing bromodeoxyuridine (BrdU) into contact with a cell sample (according to the supplier's recommendations; BrdU FlowKit; BD Pharmingen; Cat.: 557892);
B1. denaturing the DNA by the action of a deoxyribonuclease (DNase, BrdU FlowKit; BD Pharmingen; Cat.: 557892);
C1. labelling the BrdU incorporated into the DNA with an anti-BrdU antibody conjugated to a fluorochrome emitting a signal different than the signal of the other markers used, preferentially said fluorochrome is APC;
D1. detecting, by flow cytometry, said cells labeled with said BrdU;
E1. determining the percentage of tumor cells and of normal plasma cells in the S phase.

Preferentially, the method for determining the proliferation index of the tumoral plasma cells according to the invention comprises the following steps:
A2. bringing ethynyldeoxyuridine (EdU) into contact with a cell sample (according to the supplier's recommendations; Click-It® Plus EdU Alexa Fluor® 647 Flow Cytometry Assay Kit, Life Technologies, Cat.: C10635);
B2. Labeling the EdU with a fluorochrome emitting a signal different than the signal of the other markers used, preferentially said fluorochrome is Alexa Fluor® 647;
C2. detecting, by flow cytometry, said cells labeled with said EdU;
D2. determining the percentage of tumor cells and of normal plasma cells in the S phase.

Preferentially, the method for determining the proliferation index of the tumoral plasma cells according to the invention also comprises an additional step of evaluating the diploidy consisting in adding DAPI between steps C1 and D1 or B2 and C2.

Preferentially, the method for determining the proliferation index of the tumoral plasma cells according to the invention comprises the following steps:
A3. bringing bromodeoxyuridine (BrdU) into contact with a cell sample (according to the supplier's recommendations; BrdU FlowKit; BD Pharmingen; Cat.: 557892);
B3. denaturing the DNA via the action of a deoxyribonuclease (DNase, BrdU FlowKit; BD Pharmingen; Cat.: 557892);
C3. labeling the BrdU incorporated into the DNA by binding an anti-BrdU antibody conjugated to a fluorochrome emitting a signal different than the signals of the other markers used, preferentially said fluorochrome is APC;
D3. bringing DAPI into contact with the cell sample;
E3. detecting, by a flow cytometry, said cells labeled with said BrdU and DAPI;
F3. determining the diploidy and the percentage of tumor cells and of normal plasma cells in the S phase.

Preferentially, the method for determining the proliferation index of the tumoral plasma cells according to the invention comprises the following steps:
A4. bring ethynyldeoxyuridine (EdU) into contact with a cell sample (according to the supplier's recommendations; Click-It® Plus EdU Alexa Fluor® 647 Flow Cytometry Assay Kit, Life Technologies, Cat.: C10635);
B4. Labeling the EdU with a fluorochrome emitting a signal different than the signal of the other markers used, preferentially said fluorochrome is Alexa Fluor® 647;
C4. bringing DAPI into contact with the cell sample;
D4. detecting, by flow cytometry, said cells labeled with said EdU and DAPI;
E4. determining the diploidy of the tumor cells and of the normal plasma cells.

The proportion of tumoral plasma cells relative to the other leukocytes gives a picture of the cell environment, but this proportion is influenced by the variations in the other leukocyte populations.

Quantification of the tumor cells gives, for its parts, an evaluation of the tumor mass. This quantification can be carried out by analyzing a known volume by flow cytometry, using counting particles, such as fluorescent polystyrene beads (for example sold by Coulter, FlowCyto Standard Corporation, Polysciences, Sperotech, BD TrueCounts, DakoCytomation, etc.), introduced in a known amount into a known volume of sample, or else indirectly by the proportion of tumoral plasma cells relative to an otherwise quantified population (for example a number of total leukocytes of the sample, resulting from an automated counting device).

Thus, according to one particular embodiment, the method for detecting, by flow cytometry, the presence of normal plasma cells and of tumoral plasma cells in a sample of cells from a patient according to the invention also comprises:
a step of determining or receiving the amount of a cell population or a group of cell populations per unit of volume;
determining the proportion of the plasma cell populations relative to the population or group of populations quantified;
quantifying the plasma cell populations per unit of volume.

The expression “receiving the amount of a population” is intended to mean an action which makes it possible to provide the system with information regarding the amount of cells present in the sample to be analyzed or already analyzed (for example: quantification of the leukocytes). This action can for example consist of a computer transmission, or an input by the operator.

According to one particular embodiment, said patient is considered to be at risk of developing a monoclonal gammopathy when the number of tumoral plasma cells per unit of volume is greater than a predefined threshold value. This “predefined threshold value” is determined from results obtained by means of the method according to the invention from healthy and/or unwell control subjects. According to one particular embodiment of the present invention, the predefined threshold values according to the invention are parametrizable.

A subject of the present invention is also a method for diagnosing or assisting in diagnosing, in vitro, or monitoring monoclonal gammopathy, using a sample of cells from a patient, comprising the following steps:
1) carrying out the method according to the invention, and
2) demonstrating the risk of developing a monoclonal gammopathy:
   a) if the proportion of tumoral plasma cells among the total leukocyte cells, according to the results of analysis of step d) of the method according to the invention, exceeds a predefined threshold; and/or
   b) if the number of tumoral plasma cells per unit of volume exceeds a predefined threshold.

A subject of the present invention is also a method for diagnosing or assisting in diagnosing, in vitro, monoclonal gammopathy using a sample of cells of a patient, wherein steps 1) and 2) according to the invention are automated.

A subject of the present invention is also a method for monitoring, in vitro, the progression of the disease of a patient suffering from a monoclonal gammopathy using a first and a second sample of cells from the patient, comprising the following steps:
i) carrying out the detection method according to the invention in a first sample of cells from the patient,
ii) carrying out the detection method according to the invention in a second sample of cells from the patient, said second sample having been taken after said first sample was taken, and
iii) comparing the analysis of the results of step d) obtained with the first and the second sample, and
iv) monitoring the progression of said monoclonal gammopathy (tumor mass, proliferation, monoclonality, etc.) of said patient according to the results of the analysis of step iii).

A subject of the present invention is also a method for monitoring, in vitro, the progression of the disease of a patient suffering from a monoclonal gammopathy, using a first and a second sample of cells from the patient, characterized in that steps i), ii), iii) and iv) are automated.

The present invention also relates to a composition comprising at least:
a κ chain marker and a λ chain marker, emitting a different signal, and
a marker chosen from: a CD38 marker or a CD138 marker, said marker emitting a signal different than the first two signals, and
one or more CD marker(s), preferentially at least two CD markers, specific for normal plasma cells, selected from the CD19, CD27, CD45, CD81, CD5L, CD11a, CD16b, CD24, CD36, CD52, CD68, CD79a, CD80, CD82, CD84, CD99 and CD163 markers, preferentially the CD19, CD27, CD45 and CD81 markers, more preferentially a CD19 marker and a CD27 marker, each of these one or more marker(s) emitting a signal, in particular one and the same signal otherwise known as “single detectable signal”, different than the first three signals, and
one or more CD marker(s), preferentially at least two CD markers, more preferentially at least three CD markers, specific for tumoral plasma cells, selected from the CD56, CD117, CD200, CD20, CD28, CD1D, CD2BP2, CD32c, CD47, CD59, CD109, CD229, CD300a and CD320 markers, preferentially the CD56, CD117, CD200, CD20 and CD28 markers, more preferentially a CD56 marker, a CD117 marker and a CD200 marker, each of these one or more marker(s) emitting a signal, in particular one and the same signal otherwise known as “single detectable signal”, different than the first four signals.

Advantageously, the composition according to the invention comprises at least:
a κ chain marker and a λ chain marker, emitting a different signal; and
a marker chosen from: a CD38 marker and a CD138 marker, said marker emitting a signal different than the first two signals; and
a CD19 marker and a CD27 marker, said markers emitting a signal, in particular one and the same signal otherwise known as “single detectable signal”, different than the first three signals; and
a CD56 marker, a CD117 marker and a CD200 marker, said markers emitting a signal, in particular one and the same signal otherwise known as “single detectable signal”, different than the first four signals.

According to one advantageous embodiment, the composition according to the invention also comprises one or more marker(s) for tumoral plasma cells proliferation according to the invention, emitting a signal different than each of the signals of the other markers present in said composition.

The subject of the present invention is also the use of a composition comprising at least:
a κ chain marker and a λ chain marker, emitting a different signal; and
a marker chosen from: a CD38 marker and a CD138 marker, said marker emitting a signal different than the first two signals; and
one or more CD marker(s), preferentially at least two CD markers for normal plasma cells, selected from the CD19, CD27, CD45, CD81, CD5L, CD11a, CD16b, CD24, CD36, CD52, CD68, CD79a, CD80, CD82, CD84, CD99 and CD163 markers, preferentially the CD19, CD27, CD45 and CD81 markers, more preferentially a CD19 marker and a CD27 marker, each of these one or more marker(s) emitting a signal, in particular one and the same signal otherwise known as “single detectable signal”, different than the first three signals, and
one or more CD marker(s), preferentially at least two CD markers, more preferentially at Least three CD markers for tumoral plasma cells, selected from the CD56, CD117, CD200, CD20, CD28, CD1D, CD2BP2, CD32c, CD47, CD59, CD109, CD229, CD300a and CD320 markers, preferentially the CD56, CD117, CD200, CD20 and CD28 markers, more preferentially a CD56 marker, a CD117 marker and a CD200 marker, each of these one or more marker(s) emitting a signal, in particular one and the same signal otherwise known as “single detectable signal”, different than the first four signals,

for carrying out the method according to the invention.

Advantageously, the subject of the present invention is the use of a composition comprising at least:
a κ chain marker and a λ chain marker, emitting a different signal; and
a marker chosen from: a CD38 marker and a CD138 marker, said marker emitting a signal different than the first two signals; and
a CD19 marker and a CD27 marker, said markers emitting a signal, in particular one and the same signal otherwise known as “single detectable signal”, different than the first three signals; and
a CD56 marker, a CD117 marker, a CD200 marker, said markers emitting a signal, in particular one and the same signal otherwise known as “single detectable signal”, different than the first four signals;
   for carrying out the method according to the invention.

According to one particular embodiment, the use of a composition according to the invention also comprises one or more markers for tumoral plasma cell proliferation according to the invention, emitting a signal different than the signals of each of the other markers present in said composition.

A subject of the present invention is also a kit for carrying out the method according to the invention, comprising at least:
a κ chain marker and a λ chain marker, emitting a different signal; and
a CD38 marker or a CD138 marker, emitting a signal different than the first two signals; and
one or more CD marker(s), preferentially at least two CD markers for normal plasma cells, selected from the CD19, CD27, CD45, CD81, CD5L, CD11a, CD16b, CD24, CD36, CD52, CD68, CD79a, CD80, CD82, CD84, CD99 and CD163 markers, preferentially the CD19, CD27, CD45 and CD81 markers, more preferentially a CD19 marker and a CD27 marker, each of these one or more markers emitting a signal, in particular one and the same signal otherwise known as “single detectable signal”, different than the first three signals; and
one or more CD marker(s), preferentially at least two CD markers, more preferentially at least three CD markers for tumoral plasma cells, selected from the CD56, CD117, CD200, CD20, CD28, CD1D, CD2BP2, CD32c, CD47, CD59, CD109, CD229, CD300a and CD320 markers, preferentially the CD56, CD117, CD200, CD20 and CD28 markers, more preferentially a CD56 marker, a CD117 marker and a CD200 marker, each of these one or more markers emitting a signal, in particular one and the same signal otherwise known as “single detectable signal”, different than the first four signals.

According to one preferred embodiment, the kit for carrying out the method according to the invention comprises at least:
a κ chain marker and a λ chain marker, emitting a different signal; and
a CD38 marker or a CD138 marker, emitting a signal different than the first two signals; and
a CD19 marker and a CD27 marker, emitting a signal, in particular one and the same signal otherwise known as “single detectable signal”, different than the first three signals; and
a CD56 marker, a CD117 marker and a CD200 marker, emitting a signal, in particular one and the same signal otherwise known as “single detectable signal”, different than the first four signals.

According to one particular embodiment, the kit according to the invention also comprises one or more markers for tumoral plasma cell proliferation according to the invention, emitting a signal different than each of the other markers present in said kit.

Advantageously, the kit according to the invention also comprises at least one cell proliferation marker chosen from:
base analogs of BrdU type and an anti-BrdU antibody conjugated to APC, or EdU and an azide coupled to a fluorochrome, preferentially Alexa Fluor® 647; and/or
markers for proteins which are present in the active cell cycle, for example Ki67 recognized by an anti-Ki67 antibody conjugated to a fluorochrome, preferentially Alexa Fluor 647; and/or
deoxyribonucleic acid (DNA) markers, such as DAPI, PI, Hoechst dye (33342 or 33258), etc.,
each of said cell proliferation marker(s) emitting a signal different than the signals emitted by the other markers present in said kit, in particular different than the first five signals.

According to one preferential embodiment, the kit according to the invention is characterized in that it comprises at least one cell proliferation marker chosen from:
a bromodeoxyuridine (BrdU) base analog;
a deoxyribonuclease (DNase, BrdU FlowKit; BD Pharmingen; Cat.: 557892);
a marker comprising an anti-BrdU antibody conjugated to a fluorochrome, emitting a signal different than each of the other signals emitted by the markers present in said kit;
optionally DAPI emitting a signal different than the signals of each of the other markers present in said kit;
each of said cell proliferation marker(s) emitting a signal different than the signal of each of the markers present in said kit.

According to one preferential embodiment, the kit according to the invention is characterized in that it comprises at least one cell proliferation marker chosen from:
an ethynyldeoxyuridine (EdU) base analog;
an azide coupled to a fluorochrome, emitting a signal different than each of the other signals emitted by the markers present in said kit;
optionally DAPI emitting a signal different than the signals of each of the markers present in said kit.

Other characteristics and advantages of the invention emerge in the following examples and figures:

### DESCRIPTION OF THE FIGURES

FIG. 1: Discrimination of tumoral plasma cells by successive windowing.

FIG. 2: Representation of the discrimination of the tumor plasma cells by successive windowing according to the STI method.

FIG. 3: Monitoring of the proliferation of normal and tumoral plasma cells through the use of BrdU and DAPI or of Ki67 and DAPI, using a sample from a patient suffering from multiple myeloma with a normal level of S-phase plasma cells.

FIG. 4: Monitoring of the proliferation of normal and tumoral plasma cells through the use of BrdU and DAPI or of Ki67 and DAPI using a sample from a patient suffering from multiple myeloma with a high level of S-phase plasma cells.

FIG. 5: Determination of the percentage of S-phase tumor cells through the use of DAPI and of EdU.

### EXAMPLES

### Example 1: Detection of Tumoral and Normal Plasma Cells in a Patient Suffering from MM

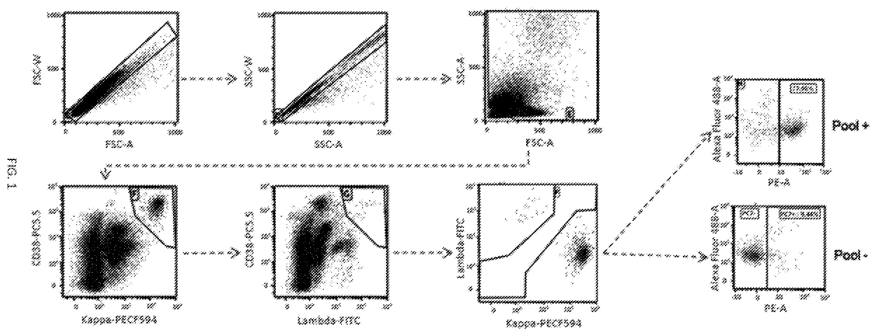

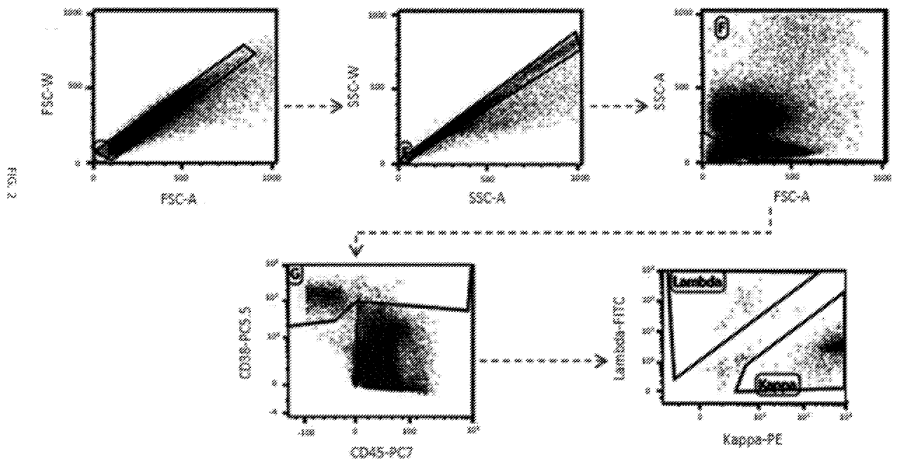

The principle of this strategy is the discrimination of the cells of interest by means of their size, their structural complexity and the presence of cell markers, or antigens, recognized by antibodies coupled to fluorochromes. There are 8 antigens characteristic of plasma cells (CD38, κ, λ, CD19, CD27, CD56, CD117 and CD200) of which the specific antibodies are coupled to 5 different fluorochromes in a single tube (CD38/PeCy5.5, κ/PECF594, λ/FITC, (CD19, CD27)/PeCy7, (CD56, CD117, CD200)/PE). This strategy can be carried out in half a day. The anti-CD38, anti-kappa and anti-lambda antibodies are used to discriminate the total plasma cells from the other leukocytes and two groups of antibodies make it possible to identify the tumor cells. The negative group contains the anti-CD19 and anti-CD27 antibodies and the positive group contains the anti-CD56, anti-CD117 and anti-CD200 antibodies. FIG. 1 represent the various windowing operations carried out in order to identify the plasma cells. The first step consists in eliminating the multiplets by means of the pulse shape analysis on the FSC and SSC channels. The debris is then eliminated on the FSC/SSC matrix. The cells which strongly express either CD38 and κ, or CD38 and λ, are then selected on the CD38/κ or CD38/λ matrices, respectively. The events on the diagonal of the κ/λ matrix are eliminated. After having discriminated the plasma cell population, the tumor cells are identified by means of the positive and negative groups.

### Preparation of the Sample

Following the taking of the bone marrow sample, said bone marrow must be filtered in order to remove any bone debris. The red blood cells are then lysed with an NH₄Cl lysis (ratio) and the debris from this is to a large extent eliminated by centrifugation. The membrane antibodies coupled to their respective fluorochrome CD56-PE (Beckman Coulter; Cat.: A07788), CD117-PE (Beckman Coulter; Cat.: IM2732), CD200-PE (BD Pharmingen; Cat.: 552475) (positive group), CD19-PeCy7 (Beckman Coulter; Cat.: IM3628), CD27-PeCy7 (Beckman Coulter; Cat.: A54823) (negative group), CD38-PeCy5.5 (Beckman Coulter; Cat.: A70205) are directly deposited in the cell preparation according to the producer's recommendations. After incubation for 20 minutes at 4° C. and washing with PBS, the cells are fixed (Intraprep kit; Beckman Coulter; Cat.: A07803). Said cells are then washed with PBS in order to remove all of the fixing solution. Finally, the cells are permeabilized (Intraprep kit; Beckman Coulter; Cat.: A07803) and labeled with the pairs consisting of intracytoplasmic antibodies/fluorochromes κ-PE-CF594 (BD Pharmingen; Cat.: 562620) and λ-FITC (BD Pharmingen; Cat.: 555796) according to the producer's recommendations. After incubation for 20 minutes at 4° C. and washing with PBS, the sample is analyzed by means of the flow cytometry device (BD LSRFORTESSA X-20, BD Biosciences).

### Analysis

### Elimination of the Multiplets

Firstly, the cells which pass through the measurement window at the same time are eliminated using the FSC and SSC parameters which correspond, respectively, to the size and to the structural complexity of the cell. For these two parameters, the matrix of height and area of the measurement peak is used and the events departing from the diagonal of this matrix are eliminated. This is because the height and area values are proportional and the absence of this proportionality (points outside the diagonal) reflects the presence of doublets or even multiplets.

### Elimination of the Debris

The cell debris due to the lysis, mainly the red blood cell debris, is then eliminated. The FSC/SSC matrix is used to select all of the leukocytes and to eliminate the cell debris having a small size and a structure which is not very complex.

### Selection of the Total Plasma Cells

The selection of the total plasma cells is carried out by means of three markers, making it possible to detect CD38 and the two intracytoplasmic proteins κ and λ. The plasma cells are cells strongly expressing the CD38 marker on the surface of their membrane. These cells are capable of producing antibodies composed of (κ or λ) light chains and of heavy chains. Each plasma cell can only produce one type of light chain and the proportion of plasma cells termed “kappa” or “Lambda” plasma cells is ⅔ and ⅓, respectively. In the patients suffering from multiple myeloma, the proliferation of a κ or λ clone will induce an imbalance of the κ/λ ratio. The plasma cells are selected on the CD38/κ and CD38/λ matrices independently.

### Elimination of the κ/λ Events

The events which are on the diagonal of the κ and λ dimensions are eliminated. Indeed, plasma cells can secrete in the κ light chain or the λ light chain throughout their life, but never both.

### Tumoral/Normal Plasma Cell Discrimination

Once the total plasma cells have been selected, the tumoral plasma cells must be discriminated with respect to the normal plasma cells by selecting the cells expressing a marker of the positive group (CD56/CD117/CD200) measured on the PE channel and/or having a loss of expression of one of the markers of the negative group (CD19/CD27) measured on the PE-Cy7 channel.

### Example 2: Comparison of the Strategy According to the Invention with a Known Prior Art Strategy

The strategy developed and carried out by the “Suivi des thérapie innovant” [“Innovative therapy monitoring” ] laboratory and described in the prior art (Caraux et al.; 2012), that will be referred to herein as “STI strategy”, calls for 10 antibodies directed against antigens characteristic of plasma cells (CD38, CD45, CD19, CD20, κ, λ, CD27, CD56, CD117 and CD200) coupled to 7 different fluorochromes distributed into 4 independent tubes.

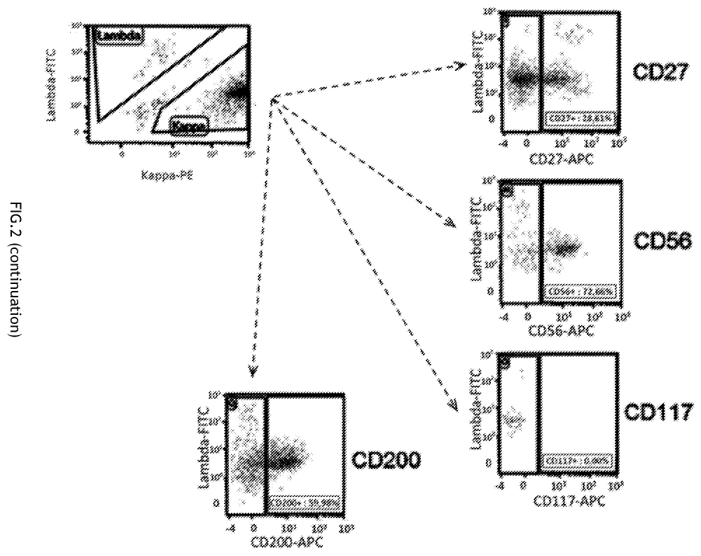

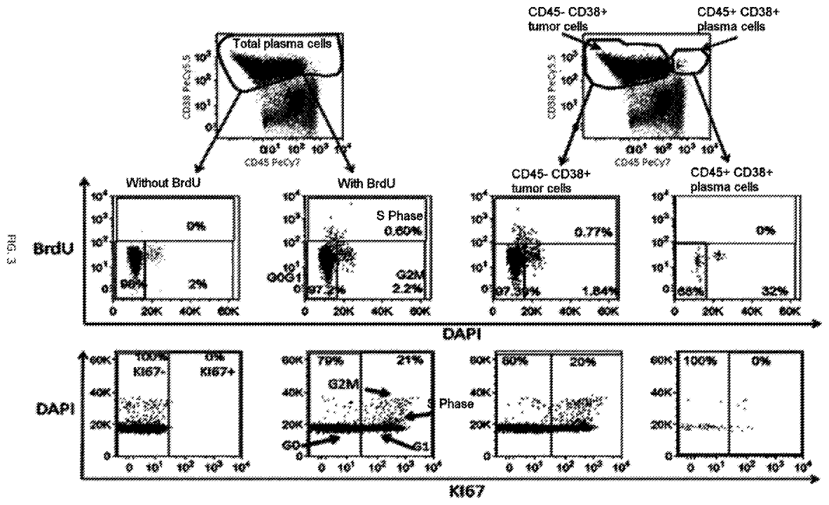

FIG. 2 represents the successive windowing operations of the STI strategy. The first step consists in eliminating the multiplets (several cells pass through the detection window at the same time) by means of FSC and SSC dimensions. The debris, of very small sizes (FSC) and granulosities (SSC), is then eliminated on the FSC/SSC matrix. All the cells strongly expressing CD38, such as the plasma cells, are then selected on the CD38/CD45 cytogram. The κ/λ events on the cytogram diagonal are then eliminated. This is because plasma cells secrete either the κ light chain or the λ light chain. After having identified the population of total plasma cells in each of the 4 tubes, the tumor cells are differentiated from the normal plasma cells by means of the markers which make it possible to protect CD27, CD56, CD117 and CD200, through loss of expression of the first one and/or expression of the subsequent ones. This prior art method thus uses seven colors, as many detectors, two lasers and four to five tubes, therefore a high and expensive number of devices and reagents. In addition, the use of such a method requires the knowhow of an expert in cytometry for the preparation, acquisition, compensation and analysis of the data. In comparison, the method according to the invention (example 1) is simpler to carry out and more economical: it can be carried out with five detectors, a single laser and a single tube, and does not require the intervention of an expert in cytometry for interpretation of the results.

### Example 3. Determination of the Percentage S-Phase Tumor Cells

The BrdU analysis makes it possible to determine the percentage of S-phase (synthesis phase) tumor cells. DAPI (4′,6′-diamidino-2-phenylindole, a fluorescent molecule capable of strongly binding to the adenine (A) and thymidine (T) bases of DNA) is used to quantify the DNA (2N/4N).

### Materials and Methods

In order to monitor cell proliferation, the APC BrdU Flow Kit was used according to the recommendations of the supplier (BD Pharmingen, Cat.: 557892).

Subsequent to the protocol described in example 1, 350 μl of a DAPI/PermWash mixture at 2 μl/ml were added to the cell solution and then incubated for 20 minutes at 4° C. The cell solution was then washed with PBS and then taken up in PBS, before being analyzed by flow cytometry (CyAn™ ADP cytometer—Beckman Coulter).

### Results

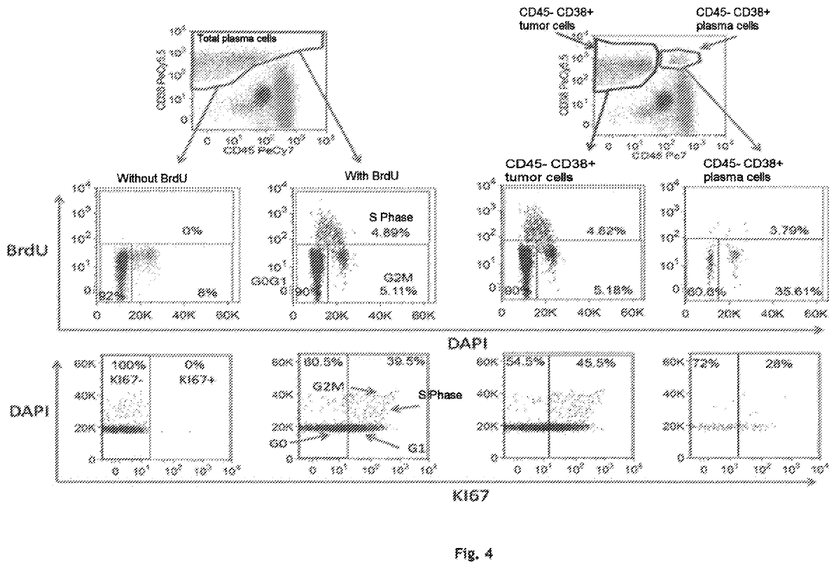

Patient suffering from multiple myeloma with a normal level of S-phase plasma cells (0.60%) (FIG. 4).

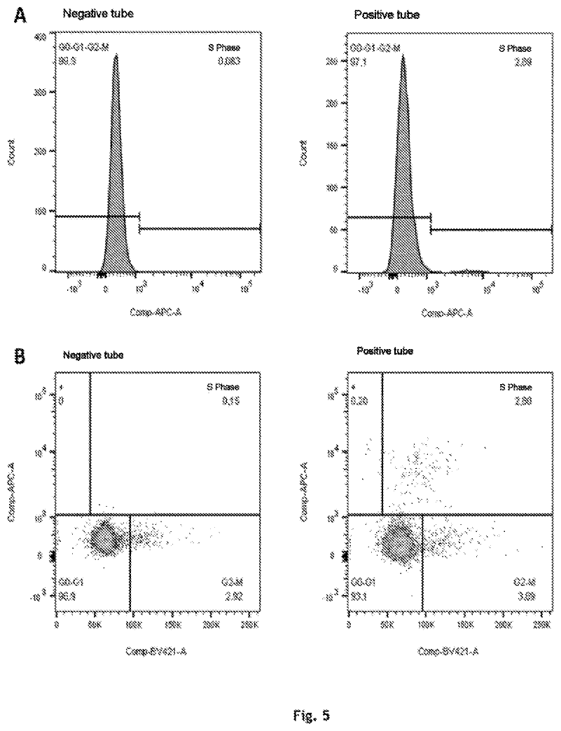

Patient suffering from multiple myeloma with a high level of S-phase plasma cells (4.89%) (FIG. 5).

### Example 4. Determination of the Percentage of Cells Engaged in the Cell Cycle (Non-Quiescent Cells)

Ki67 makes it possible to quantify the percentage of cells engaged in the cell cycle (non-quiescent cells). DAPI (4′,6′-diamidino-2-phenylindole, a fluorescent molecule capable of binding strongly to the adenine (A) and thymidine (T) bases of DNA) is used to quantify the DNA (2N/4N).

### Materials and Methods

Two tubes are used, a negative control with the APC isotype (Beckman Coulter, Ref: IM2475U) added to the cell solution at the time of the surface antibody labeling, and a Ki67 tube positive with the anti-Ki67 coupled to Alexa Fluor 647 (BD Pharmingen, Cat.: 558615) added to the cell solution at the time of the intracytoplasmic antibody labeling.

Subsequent to the preceding protocol, 350 μl of a DAPI/PermWash mixture at 2 μl/ml were added to the cell solution and then incubated for 20 minutes at 4° C. The cell solution is then washed with PBS then taken up in PBS, before being analyzed by flow cytometry (CyAn™ ADP cytometer—Beckman Coulter).

### Results

Patient suffering from multiple myeloma with a level of cells engaged in the cell cycle (non-quiescent cells) (21%) (FIG. 4).

Patient suffering from multiple myeloma with a level of cells engaged in the cell cycle (non-quiescent cells) (39.5%) (FIG. 5).

### Example 5. Determination of the Percentage of S-Phase Tumor Cells Using DAPI and EdU

### Materials and Methods

To monitor the cell proliferation, the Click It® Plus EdU Alexa Fluor® 647 Flow Cytometry Assay Kit was used according to the recommendations of the supplier (Life Technologies, Cat.: C10635).

Subsequent to the preceding protocol, 350 μl of a DAPI/PermWash mixture at 2 μl/ml were added to the cell solution and then incubated for 20 minutes at 4° C. The cell solution is then washed with PBS then taken up in PBS, before being analyzed by flow cytometry (BD LSRFortessa™ X-20 cytometer—BD Biosciences).

### Results

The monitoring of the plasma cell proliferation can be carried out in two dimensions (DAPI vs EdU) or in one dimension (EdU histogram).

The results presented in FIG. 5 show that, by using one or two dimensions, it is possible to identify, respectively, 2.89% and 2.80% of S-phase plasma cells in the presence of EdU (positive tube) in a patient diagnosed with multiple myeloma.

### LITERATURE REFERENCES

1. Caraux, A., Klein, B., Paiva, B., Bret, C., Schmitz, A., Fuhler, G. M., Bos, N. A., Johnsen, H. E., Orfao, A., Perez-Andres, M., et al. (2010). Circulating human B and plasma cells. Age-associated changes in counts and detailed characterization of circulating normal CD138− and CD138+ plasma cells. Haematologica 95, 1016-1020.
2. Caraux, A., Vincent, L., Bouhya, S., Quittet, P., Moreaux, J., Requirand, G., Veyrune, J.-L., Olivier, G., Cartron, G., Rossi, J.-F., et al. (2012). Residual malignant and normal plasma cells shortly after high dose melphalan and stem cell transplantation. Highlight of a putative therapeutic window in Multiple Myeloma? Oncotarget 3, 1335-1347.
3. Martinez-Lopez, J., Lahuerta, J. J., Pepin, F., González, M., Barrio, S., Ayala, R., Puig, N., Montalban, M. A., Paiva, B., Weng, L., et al. (2014). Prognostic value of deep sequencing method for minimal residual disease detection in multiple myeloma. Blood 123, 3073-3079.
4. Mateo, G., Montalban, M. A., Vidriales, M.-B., Lahuerta, J. J., Mateos, M. V., Gutierrez, N., Rosinol, L., Montejano, L., Bladé, J., Martinez, R., et al. (2008). Prognostic Value of Immunophenotyping in Multiple Myeloma: A Study by the PETHEMA/GEM Cooperative Study Groups on Patients Uniformly Treated With High-Dose Therapy. J. Clin. Oncol. 26, 2737-2744.
5. Paiva, B., Vidriales, M.-B., Cerveró, J., Mateo, G., Perez, J. J., Montalban, M. A., Sureda, A., Montejano, L., Gutierrez, N. C., Coca, A. G. de, et al. (2008). Multiparameter flow cytometric remission is the most relevant prognostic factor for multiple myeloma patients who undergo autologous stem cell transplantation. Blood 112, 4017-4023.
6. Paiva, B., Vidriales, M.-B., Montalban, M.-Á., Pérez, J. J., Gutierrez, N. C., Rosinol, L., Martinez-López, J., Mateos, M.-V., Cordón, L., Oriol, A., et al. (2012). Multiparameter flow cytometry evaluation of plasma cell DNA content and proliferation in 595 transplant-eligible patients with myeloma included in the Spanish GEM2000 and GEM2005<65y trials. Am. J. Pathol. 181, 1870-1878.
7. Paiva, B., Chandia, M., Puig, N., Vidriales, M.-B., Perez, J. J., Lopez-Corral, L., Ocio, E. M., Garcia-Sanz, R., Gutierrez, N. C., Jimenez-Ubieto, A., et al. (2014). The prognostic value of multiparameter flow cytometry minimal residual disease assessment in relapse multiple myeloma. Haematologica.
8. Rawstron, A. C., Child, J. A., de Tute, R. M., Davies, F. E., Gregory, W. M., Bell, S. E., Szubert, A. J., Navarro-Coy, N., Drayson, M. T., Feyler, S., et al. (2013). Minimal residual disease assessed by multiparameter flow cytometry in multiple myeloma: impact on outcome in the Medical Research Council Myeloma IX Study. J. Clin. Oncol. Off. J. Am. Soc. Clin. Oncol. 31, 2540-2547.

1. A method for detecting, by flow cytometry, the presence of normal plasma cells and of tumoral plasma cells in a sample of cells from a patient, said method comprising:
a) bringing said sample of cells into contact with:
a κ chain marker, a λ chain marker, and a marker of: a CD38 marker or a CD138 marker, each of the three markers emitting a different signal, and
one or more tumoral plasma cell marker(s), each of these one or more marker(s) emitting a signal different than said first three signals, and
one or more normal plasma cell CD marker(s), each of these one or more marker(s) emitting a signal different than the first four signals,
b) detecting the plasma cells comprising detecting:
a positive signal with a CD38 marker or a CD138 marker, and
a positive signal with a κ chain marker or a λ chain marker,
c) detecting the tumoral plasma cells among the plasma cells detected in step b) having at least one of the following criteria:
a positive signal with at least one marker specific for tumoral plasma cells, and/or
a negative or significantly reduced signal with at least one marker for normal plasma cells,
d) analyzing the results obtained during b) and c) and determining the presence of normal plasma cells and of tumoral plasma cells.

2. The method of claim 1, wherein the one or more normal plasma cell CD marker(s) is CD19, CD27, CD45, CD81, CD5L, CD11a, CD16b, CD24, CD36, CD52, CD68, CD79a, CD80, CD82, CD84, CD99 and/or CD163, and
wherein the one or more tumoral plasma cell marker(s) is CD56, CD117, CD200, CD20, CD28, CD1D, CD2BP2, CD32c, CD47, CD59, CD109, CD229, CD300a and/or CD320.

3. The method of claim 1, wherein the CD38 or CD138 marker is respectively an anti-CD38 or anti-CD138 antibody conjugated to the PE-Cy5.5 fluorochrome, the κ chain marker is the anti-κ chain antibody conjugated to the PE-CF594 fluorochrome and the λ chain marker is an anti-λ chain antibody conjugated to the FITC fluorochrome.

4. The method of claim 2, wherein the normal plasma cell CD marker(s) are CD19 and CD27 markers and the CD19 and CD27 markers are anti-CD19 and anti-CD27 antibodies conjugated to the PE-Cy7 fluorochrome, and the tumoral plasma cell marker(s) are CD56, CD117 and CD200 markers and the CD56, CD117 and CD200 markers are anti-CD56, anti-CD117 and anti-CD200 antibodies conjugated to the PE fluorochrome.

5. The method of claim 1, further comprising determining a proliferation index of the tumoral plasma cells, comprising:
a) bringing said cell sample into contact with one or more cell proliferation marker(s), emitting a signal different than the other markers used;
b) detecting the proliferating tumor plasma cells by analysis of the signal emitted by said proliferation marker(s);
c) determining the percentage of tumor cells and of normal plasma cells which are in the S phase; and
d) optionally determining the diploidy (G0/G1 or G2/M) when at least one of said cell proliferation marker(s) is a DNA marker.

6. The method of claim 1, wherein the cell sample is from the bone marrow, the blood, the serum, a blood extract, PBMCs, the cerebrospinal fluid, the pleural fluid or the lymph nodes of the patient.

7. The method of claim 1, wherein said patient is suffering from monoclonal gammopathy.

8. A method for diagnosing, in vitro, or monitoring monoclonal gammopathy, using a sample of cells from a patient, comprising:
1) performing the method of claim 1, and
2) demonstrating a risk of developing a monoclonal gammopathy:
a) if the proportion of tumoral plasma cells among the total leukocyte cells, according to the results of the analysis of d), exceeds a threshold predefined on the basis of results obtained on healthy control subjects, and/or
b) if the number of tumoral plasma cells per unit of volume exceeds a predefined threshold.

9. A method for monitoring, in vitro, the progression of the disease of a patient suffering from a monoclonal gammopathy, using a first and a second sample of cells from the patient, comprising:
i) performing the detection method of claim 1 in a first sample of cells from the patient,
ii) performing the detection method of claim 1 in a second sample of cells from the patient, said second sample having been taken after said first sample was taken, and
iii) comparing the analysis of the results of d) obtained with the first and the second sample, and
iv) monitoring the progression of said monoclonal gammopathy of said patient according to the results of the analysis of iii).

10. A composition comprising at least:
a κ chain marker and a λ chain marker, each emitting a different signal, and
a CD38 marker or a CD138 marker, said marker emitting a signal different than the first two signals, and
one or more CD marker(s) specific for normal plasma cells, wherein the one or more CD marker(s) specific for normal plasma cells are CD19, CD27, CD45, CD81, CD5L, CD11a, CD16b, CD24, CD36, CD52, CD68, CD79a, CD80, CD82, CD84, CD99 and CD163 markers, each of these one or more marker(s) emitting a signal different than the first three signals, and
one or more CD marker(s) specific for tumoral plasma cells, wherein the one or more CD marker(s) specific for tumoral plasma cells are CD56, CD117, CD200, CD20, CD28, CD1D, CD2BP2, CD32c, CD47, CD59, CD109, CD229, CD300a and/or CD320 markers, each of these one or more marker(s) emitting a signal different than the first four signals.

11. (canceled)

12. A kit for carrying out the method of claim 1, comprising at least:
a κ chain marker and a λ chain marker, each emitting a different signal, and
a CD38 marker or a CD138 marker emitting a signal different than the first two signals; and
one or more CD marker(s) for normal plasma cells, wherein the one or more CD marker(s) for normal plasma cells are CD19, CD27, CD45, CD81, CD5L, CD11a, CD16b, CD24, CD36, CD52, CD68, CD79a, CD80, CD82, CD84, CD99 and/CD163 markers, each of these one or more marker(s) emitting a signal different than the first three signals, and
one or more CD marker(s) for tumoral plasma cells, wherein the one or more CD marker(s) for tumoral plasma cells are CD56, CD117, CD200, CD20, CD28, CD1D, CD2BP2, CD32c, CD47, CD59, CD109, CD229, CD300a and/or CD320 markers, each of these one or more marker(s) emitting a signal different than the first four signals.

13. The kit as claimed in claim 12, further comprising at least one cell proliferation marker of:
base analogs of BrdU type and an anti-BrdU antibody conjugated to APC or EdU and an azide coupled to a fluorochrome; and/or
markers for proteins of the active cell cycle, for example an anti-Ki67 antibody conjugated to a fluorochrome; and/or
deoxyribonucleic acid (DNA) markers, such as DAPI, PI or Hoechst dye (33342 or 33258), each of said cell proliferation marker(s) emitting a signal different than the signals emitted by the other markers present in said kit.

14. The method of claim 1, wherein CDs specific for normal plasma cells are CD19, CD27, CD45 and/or CD81, and CDs specific for tumoral plasma cells are CD56, CD117, CD200, CD20 and/or CD28.

15. The method of claim 1, wherein CDs specific for normal plasma cells are CD19, and/or CD27, and CDs specific for tumoral plasma cells are CD56, CD117, and/or CD200.

16. The composition of claim 10, wherein the CD marker(s) specific for normal plasma cells are CD19, CD27, CD45 and/or CD81, and the CD marker(s) specific for tumoral plasma cells are CD56, CD117, CD200, CD20 and/or CD28.

17. The composition of claim 10, wherein the CD marker(s) specific for normal plasma cells are CD19 and CD27, and the CD marker(s) specific for tumoral plasma cells are CD56, CD117, and CD200.

18. The kit of claim 12, wherein the CD marker(s) for normal plasma cells are CD19, CD27, CD45 and/or CD81, and the CD marker(s) for tumoral plasma cells are CD56, CD117, CD200, CD20 and/or CD28.

19. The kit of claim 12, wherein the CD marker(s) for normal plasma cells are CD19 and CD27, and the CD marker(s) for tumoral plasma cells are CD56, CD117, and CD200.
